(19) United States
(12) Patent Application Publication
(10) Pub. No.: US 2006/0247493 A1
(43) Pub. Date: Nov. 2, 2006

(54) Vibrating Sex Auxiliary Ring Capable of Preventing Power From Accidentally Being Turned On

(76) Inventor: Chih-Hung Chen, Chin-Men Hsien (TW)

Correspondence Address: NORTH AMERICA INTELLECTUAL PROPERTY CORPORATION, P.O. BOX 506, MERRIFIELD, VA, 22116, US

(21) Appl. No.: 11/163,256

(22) Filed: Oct. 12, 2005

(30) Foreign Application Priority Data
Apr. 27, 2005 (TW) 094113463

(51) Int. Cl. A61F 5/00

(52) U.S. Cl. 600/38

(57) ABSTRACT

A vibrating sex auxiliary ring capable of preventing power from accidentally being turned on includes a ring and a vibrator formed on the ring. The vibrator includes a housing, a motor formed in the housing comprising a bias round in an axis of the motor, a battery formed in the housing comprising a first end coupled to a first power input end of the motor and a second end coupled to a conductive leaf spring that is disposed near a second power input end of the motor, and a stick comprising a first end projecting from the housing and a second end disposed between the conductive leaf spring and the second power input end of the motor in the housing for electrically connecting the conductive leaf spring and the motor when the first end of the stick is pulled out from the housing.

### BACKGROUND OF THE INVENTION

1. Field of the Invention

The present invention provides a vibrating sex auxiliary ring, and more particularly, a vibrating sex auxiliary ring capable of preventing power from accidentally being turned on.

2. Description of the Prior Art

As people's sexual concepts change, venereal diseases, or sexually transmitted diseases, caused by sexual activity have increased. Researches reveal that wearing a condom correctly can prevent venereal diseases effectively. However, a lot of people, especially young people 15 to 35 years old, still neglect to wear condoms in sexual behaviors, such that infection of venereal diseases (including AIDS) cannot be decreased.

In order to encourage people to wear condoms, the prior art provides a vibrating ring, which needs to be combined with a condom. Please refer to FIG. 1, which illustrates a schematic diagram of a prior art vibrating ring 10. The vibrating ring 10 includes a vibrator 12 and a ring 14. When a stick 16 is pressed down along an arrow 18 shown in FIG. 1, a battery in the vibrator 12 provides power to a motor to rotate a bias round in an end of an axis of the motor, so as to generate vibration. Please refer to FIG. 2 further, which illustrates a schematic diagram of the vibrator 12 shown in FIG. 1. In FIG. 2, other than the stick 16, the vibrator 12 includes a battery 20, a conductive leaf spring 22, a motor 24, and a bias round 26 in front of the motor 24. One end of the battery 20 connects to a power input end of the motor 24, and the other end of the battery 20 connects to the conductive leaf spring 22. When the stick 16 is not pressed down, an insulating end in front of the stick 16 isolates the conductive leaf spring 22 from the motor 24. When the stick 16 is pressed down, the insulating end of the stick 16 moves along the arrow 18, so that the conductive leaf spring 22 connects to the motor 24, causing the motor 24 to rotate, and generating vibration with the bias round 26.

When wearing the vibrating ring 10, a user must wear a condom first for spreading lubricant on the condom. Therefore, the utility rate of condoms can be increased if the user uses the vibrating ring 10.

The prior art vibrating ring 10 starts vibration when the stick 16 is pressed down to the vibrator 12 (along the arrow 18 shown in FIG. 1). That is, once the stick 16 is pressed down, the motor 24 starts to rotate. Therefore, if the stick 16 is accidentally pressed down while the vibrating ring 10 is being transported, the battery 20 may run out of power, and a user cannot use the vibrating ring 10, which may decrease the user's desire to use the vibrating ring 10, and cause the user to neglect using a condom during sexual activity. In addition, the prior art vibrating ring 10 is not waterproof, so the vibrating ring 10 may be loose power if water gets into the power supply during sexual activity.

### SUMMARY OF THE INVENTION

It is therefore a primary objective of the claimed invention to provide a vibrating sex auxiliary ring capable of preventing power from accidentally being turned on.

The present invention discloses a vibrating sex auxiliary ring capable of preventing power from accidentally being turned on. The vibrating sex auxiliary ring comprises a ring and a vibrator formed on the ring. The vibrator comprises a housing, a motor formed in the housing comprising a bias round in an axis of the motor, a battery formed in the housing comprising a first end coupled to a first power input end of the motor and a second end coupled to a conductive leaf spring that is disposed near a second power input end of the motor, and a stick comprising a first end projecting from the housing and a second end disposed between the conductive leaf spring and the second power input end of the motor in the housing for electrically connecting the conductive leaf spring and the motor when the first end of the stick is pulled out from the housing.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 illustrates a schematic diagram of a prior art vibrating ring.

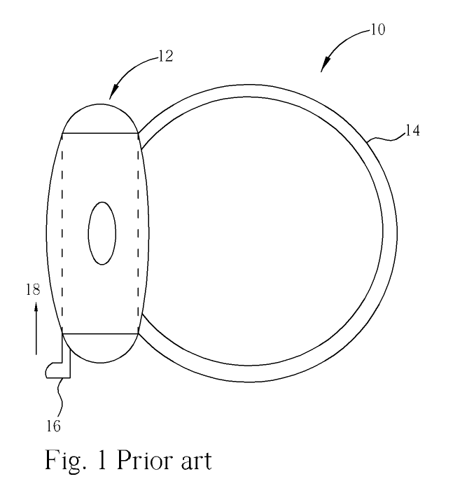

FIG. 2 illustrates a schematic diagram of the vibrator shown in FIG. 1.

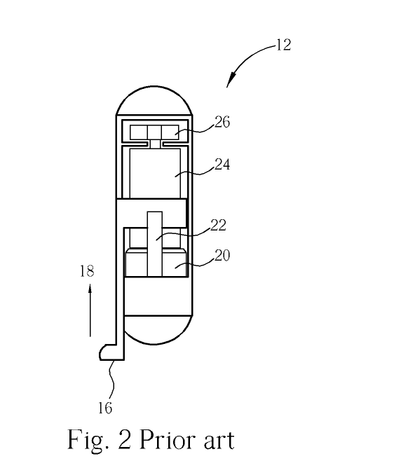

FIG. 3 illustrates a schematic diagram of a vibrating sex auxiliary ring in accordance with the present invention.

FIG. 4 illustrates a schematic diagram of the vibrator shown in FIG. 3.

FIG. 5 illustrates a schematic diagram of a vibrating sex auxiliary ring in accordance with another embodiment of the present invention.

FIG. 6 illustrates a schematic diagram of the vibrator shown in FIG. 5.

### DETAILED DESCRIPTION

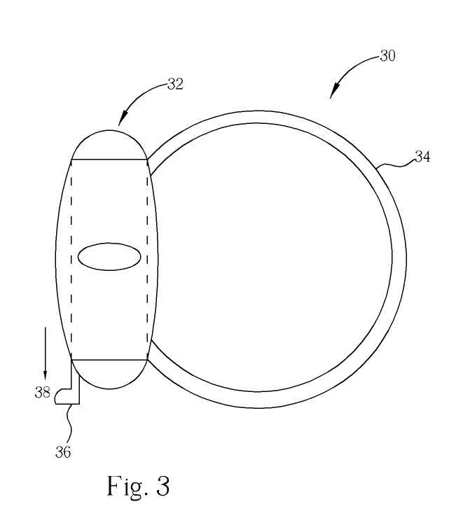

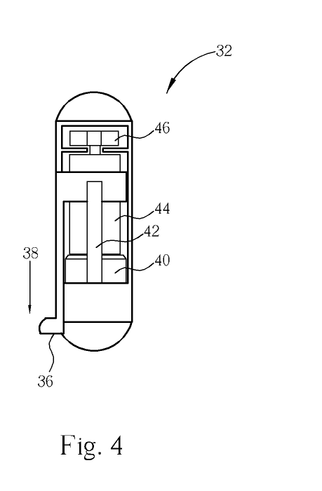

Please refer to FIG. 3, which illustrates a schematic diagram of a vibrating sex auxiliary ring 30 in accordance with the present invention. The vibrating sex auxiliary ring 30 can prevent power from accidentally being turned on, and includes a vibrator 32 and a ring 34. When a stick 36 is pulled out along an arrow 38 shown in FIG. 3, a battery in the vibrator 12 provides power to a motor to rotate a bias round in an end of an axis of the motor, so as to generate vibration. The ring 34 is made of silica gel or other elastic materials, and includes an oval-shaped bulge for massaging an erogenous zone (or clitoris) after the vibrator 32 generates vibration. Please refer to FIG. 4 further, which illustrates a schematic diagram of the vibrator 32 shown in FIG. 3. In FIG. 4, other than the stick 36, the vibrator 32 includes a battery 40, a conductive leaf spring 42, a motor 44, and a bias round 46 in front of the motor 44. One end of the battery 40 connects to a power input end of the motor 44, and the other end of the battery 40 connects to the conductive leaf spring 42. When the stick 36 is not pulled out, an insulating end in front of the stick 36 isolates the conductive leaf spring 42 from the motor 44. When the stick 36 is pulled out, the insulating end of the stick 36 moves along the arrow 38, so that the conductive leaf spring 42 connects to the motor 44, causing the motor 44 to rotate, and generate vibration with the bias round 46.

Therefore, the present invention vibrating sex auxiliary ring 30 starts the vibrator 32 by pulling the stick 36 out, so as to prevent power from accidentally being turned on while the sex auxiliary ring 30 is being transported.

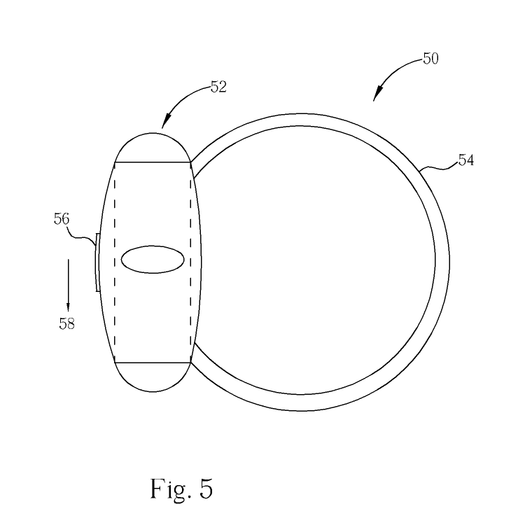

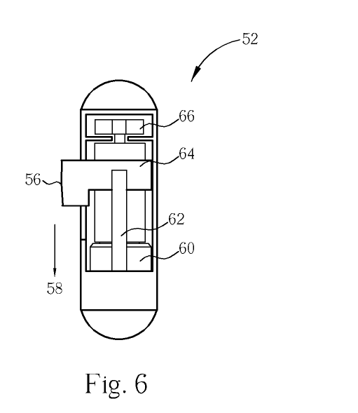

Please refer to FIG. 5, which illustrates a schematic diagram of a vibrating sex auxiliary ring 50 in accordance with another embodiment of the present invention. The vibrating sex auxiliary ring 50 can prevent power from accidentally being turned on, and includes a vibrator 52 and a ring 54. The ring 54 is made of silica gel or other elastic materials, and includes an oval-shaped bulge for massaging an erogenous zone (or clitoris) after the vibrator 52 generates vibration. When a stick 56 is pulled out along an arrow 58 shown in FIG. 5, a battery in the vibrator 52 provides power to a motor to rotate a bias round in an end of an axis of the motor, so as to generate vibration. The ring 54 wraps around the stick 56, and a user can move the stick 56 through the ring 54 because of the elastic material of the ring 54. Please refer to FIG. 6 further, which illustrates a schematic diagram of the vibrator 52 shown in FIG. 5. In FIG. 6, other than the stick 56, the vibrator 52 includes a battery 60, a conductive leaf spring 62, a motor 64, and a bias round 66 in front of the motor 64. One end of the battery 60 connects to a power input end of the motor 64, and the other end of the battery 60 connects to the conductive leaf spring 62. When the stick 66 is not pulled out, an insulating end in front of the stick 56 isolates the conductive leaf spring 62 from the motor 64. When the stick 66 is pulled out, the insulating end of the stick 66 moves along the arrow 58, so that the conductive leaf spring 62 connects to the motor 64, causing the motor 64 to rotate, and generate vibration with the bias round 66.

As shown in FIG. 5, the ring 54 wraps around the stick 56 of the vibrator 52, so the vibrating sex auxiliary ring 50 not only prevents power from accidentally being turned on caused by collision or compression in the conveyance of goods, but it is also waterproof.

In summary, when wearing the vibrating sex auxiliary ring of the present invention, a user must wear a condom, so the utility rate of condoms can be increased. The present invention vibrating sex auxiliary ring can bring pleasure not only the user, but also to the user's partner, and the elasticity of the ring in the vibrating sex auxiliary ring can help the user's sex function. In addition, the present invention can prevent power from accidentally being turned on, and is waterproof.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A vibrating sex auxiliary ring capable of preventing power from accidentally being turned on, comprising:
a ring;
a vibrator formed on the ring comprising:
a housing;
a motor formed in the housing comprising a bias round in an axis of the motor;
a battery formed in the housing comprising a first end coupled to a first power input end of the motor and a second end coupled to a conductive leaf spring that is disposed near a second power input end of the motor; and
a stick comprising a first end projecting from the housing and a second end disposed between the conductive leaf spring and the second power input end of the motor in the housing for electrically connecting the conductive leaf spring and the motor when the first end of the stick is pulled out from the housing.

2. The vibrating sex auxiliary ring of claim 1, wherein the ring comprises an oval-shaped bulge.

3. The vibrating sex auxiliary ring of claim 1, wherein the stick projects from the ring.

4. The vibrating sex auxiliary ring of claim 1, wherein the ring wraps around the stick.

5. The vibrating sex auxiliary ring of claim 1, wherein the ring is made of silica gel.
